(19) United States
(12) Patent Application Publication
(10) Pub. No.: US 2024/0279590 A1
(43) Pub. Date: Aug. 22, 2024

(54) DEVICE ARRANGEMENT AND METHOD OF SEPARATING CELLS FROM A CULTURE MEDIUM IN A BIOPROCESS

(71) Applicant: Sartorius Stedim Biotech GmbH, Goettingen (DE)

(72) Inventor: Bernhard DIEL, Goettingen (DE)

(21) Appl. No.: 18/569,271

(22) PCT Filed: Jun. 14, 2022
(86) PCT No.: PCT/EP2022/066215

§ 371 Date: Dec. 12, 2023

(30) Foreign Application Priority Data
Jun. 16, 2021 (EP) 21179865.7

(51) Int. Cl. C12M 1/00; C12M 1/12

(52) U.S. Cl. CPC C12M 47/04; C12M 29/16; C12M 37/02; C12M 39/00

(57) ABSTRACT

A device arrangement for separating cells from a culture medium in a bioprocess, in particular in a biopharmaceutical process, including a storage tank for the culture medium; a filtration module having a membrane adapted to be overflowed; an intake line connecting the storage tank to an unfiltrate inlet for supplying culture medium into the filtration module; a return line which connects a retentate-side outlet of the filtration module to the storage tank for returning retentate into the storage tank; a filtrate line which is connected to a permeate-side outlet of the filtration module for discharging filtrate; a first pump for maintaining a recirculation circuit; a backflush line which leads to a permeate-side connection and/or to the permeate-side outlet for supplying a rinsing liquid into the filtration module; and a second pump for conveying the rinsing liquid into the filtration module. A method of separating cells from a culture medium.

### FIELD OF THE INVENTION

The invention relates to a device arrangement and to a method of separating cells from a culture medium in a bioprocess, in particular in a biopharmaceutical process.

### BACKGROUND

In the biopharmaceutical industry, a clear trend towards increasing efficiency and intensification of cultivation processes can be observed. Constantly improving technologies and optimized processes lead to ever smaller fermentation volumes with at the same time increasingly higher cell and protein concentrations, which must be processed in the subsequent process steps to clarify and isolate the actual target substance.

For this reason, the first clarification step of cell separation, directly after cultivation, is becoming increasingly important, as high concentrations of active ingredients are usually accompanied by high cell numbers and thus larger wet bio-masses. Conventional methods of cell separation, such as centrifugation and depth filtration and, in some cases, cross-flow filtration (tangential flow filtration, TFF), are therefore faced with ever-increasing challenges and often reach technical limits.

In addition, a steadily increasing interest in single-use process solutions can be observed in the biopharmaceutical industry, with the aim of optimizing or accelerating costly and time-consuming validation processes for stainless steel systems—at all intermediate stages on the way of the development of a new drug. The increasing acceptance of single-use technologies throughout the entire manufacturing process of biopharmaceuticals results in the constant search for new and innovative technologies and techniques to meet these challenges. The use of single-use bioreactors is also gaining ground in the fermentation process at the beginning of the process chain and is already very widely used today. The cell separation step following fermentation is usually carried out in single-use production plants with two or more depth filtration steps. However, this filtration technology is traditionally limited with regard to the concentration of the cell masses to be separated, as rapid blocking of the filter surface can hardly be counteracted. At the same time, there are currently no hermetically sealed single-use depth filtration systems that users would like to use to prevent product contact with the environment or even the operating personnel. This means that depth filter technology, which is already subject to technical limitations, is increasingly pushed to its economic limits by the intensification trend described above, in addition to its lack of practical properties.

In other branches of industry, such as the beverage industry, where there are similar problems, cross-flow filtration has proven to be a suitable way of separating cells in an efficient and at the same time economic way compared to conventional methods. Cross-flow filtration can often replace two or more traditional clarification steps in a single process step, namely centrifugation and often a subsequent multi-stage depth filtration. Hollow fiber modules having ultrafiltration or microfiltration membranes are particularly suitable for cell separation using cross-flow filtration.

Though cross-flow filtration with microfiltration membranes is widely used and well known as an established separation process, the cell fraction contained in the fermentation broth in the form of the so-called wet bio-mass and the protein yield to be achieved pose a particular challenge in the field of application of interest here. This is because with increasing separation of clarified protein filtrate (permeate) from the original fermenter solution, the concentration of the wet bio-mass present in the bioreactor increases successively and leads to an increase in viscosity in the course of filtration. This process cannot be continued indefinitely due to the technical general conditions. It is therefore important to design or optimize the technical general conditions such that the yield can be maximized.

The general conditions represent an interaction of various factors which can strongly influence the economic efficiency of a separation process. These essentially involve the geometric design of the filtration unit (here in particular a hollow fiber module), the system conditions, such as the pump system, as well as the dynamically changing liquid condition (e.g. viscosity and temperature). With increasing concentration and increasing viscosity, the free flow through the tubular channels of the hollow fiber module is no longer possible or only possible to a limited extent, so that the filtration performance can drop sharply and optimum flow over the membrane surface becomes increasingly difficult. This can go so far that the filtration can no longer be designed economically in terms of specific flow rate and pump energy input, or a total collapse in filtration performance occurs and the process has to be interrupted.

To avoid such an escalation point, controlled dilution with buffer or substrate (nutrient solution), for example, could help to keep the liquid at a viscosity level suitable for the system. However, this in turn has the disadvantage that the solution containing the target protein takes up an ever-increasing volume, which is also a disadvantage in the further purification steps downstream of the first clarification step.

In the application of primary interest here, the success or failure of the respective cell separation technology is therefore characterized not only by technical feasibility but primarily also by the protein yield to be achieved—as a value-giving component. This thus means that an optimal technology, combined with a suitable process, must be found, which enables the maximum protein yield to be achieved without negatively influencing the subsequent steps in the purification process of the biopharmaceutical target protein.

The optimum operating point described above can be achieved by first carrying out cell separation by means of cross-flow filtration in the classic manner, i.e. with separation of the protein solution and simultaneous concentration of the biomass. As soon as the separation of the clear cell-free solution reaches a critical viscosity value or a lower filtrate flow limit, dilution takes place by supplying a dilution liquid into the system. In known cross-flow filtration processes, the dilution liquid is usually supplied directly into the feed or recirculation tank, or into the cross-flow filtration circulation line.

To maintain or optimize the filtration flow through the membrane, cross-flow filtration processes have already been presented in which a backflush with filtrate which has just passed through the membrane is carried out. Corresponding systems are described, for example, in documents U.S. Pat. No. 6,544,424 B1 and WO 2018/022661 A1. In the technology also referred to as “Alternating Tangential Flow (ATF) Filtration”, the surface coating (fouling) which builds up is loosened or detached by reversing the flow direction and thus also the pressure ratios between the mean inlet pressure on the membrane inflow side and the outlet pressure on the membrane outflow side and, ideally, carried away with the retentate flow and transported away. Ideally, this allows the filtrate flow (flux) to be completely or at least partially increased back to an initial flow value or at least maintained at a high level for a longer period of time. The disadvantage of this method, however, is that backflushing with filtrate has a negative effect on the effective filtration performance and thus the efficiency of the overall system, as the filtrate which has just been returned must be guided from the inflow side of the membrane to the filtrate side again after backflushing.

### SUMMARY OF THE INVENTION

Against this background, it is the object of the invention to provide a technology for cell separation which makes it possible to achieve the highest possible yield of a biopharmaceutical target protein without negatively influencing the subsequent steps in the purification process of the protein. In particular, such a technology using microfiltration membranes, in particular in hollow fiber format, but also in other formats such as plate-frame modules, tubular modules, spiral-wound modules, etc., should allow a technically and economically optimal operating point to be set, especially with regard to product yield, product quality and degree of dilution of the solution.

This object is achieved by a device arrangement having the features of claim 1 and by a method having the features of claim 13. Advantageous and practical configurations of the device arrangement according to the invention and of the method according to the invention are given in the associated subclaims.

The device arrangement according to the invention for separating cells from a culture medium in a bioprocess, in particular in a biopharmaceutical process, comprises:
a storage tank for the culture medium, in particular a bioreactor;
a filtration module, in particular a hollow fiber module, having at least one membrane adapted to be overflowed;
an intake line which connects the storage tank to an unfiltrate inlet of the filtration module for supplying culture medium into the filtration module;
a return line which connects a retentate-side outlet of the filtration module to the storage tank for returning retentate into the storage tank;
a filtrate line which is connected to a permeate-side outlet of the filtration module for discharging filtrate;
a first pump for maintaining a recirculation circuit;
a backflush line which leads to a (different) permeate-side connection and/or to the permeate-side outlet of the filtration module for supplying a rinsing liquid into the filtration module; and
a second pump for conveying the rinsing liquid into the filtration module.

With the device arrangement according to the invention, the cell separation step can be optimally designed independently of the type of fermentation process to optimize both protein yield and process costs under dynamic, i.e. changing, general conditions. In particular, the set-up according to the invention makes it possible to advantageously combine a dilution of the culture medium required or desired during filtration with a cleaning of the membrane surface. Due to the separate backflush line and the second pump, a rinsing liquid by means of which, on the one hand, the covering layer or other residues (fouling) that form in the course of filtration can be loosened or detached from the membrane, can additionally be introduced into the recirculation circuit during filtration, to thus contribute, on the other hand, to the dilution of the circulating culture medium. The rinsing liquid can therefore fulfill several functions simultaneously, namely (i) cleaning of the membrane to counteract blockage of the filter and maintain the filtration performance as well as possible, and (ii) dilution of the culture medium to keep it at a viscosity level suitable for the system, and/or (iii) optimal control of cell nutrition during fermentation, which will be discussed in more detail later.

A buffer solution adapted to the culture medium is particularly suitable as a rinsing liquid. In conventional systems, the introduction of such a buffer solution is provided at most for the purpose of diluting the culture medium during filtration. According to the present invention, the buffer solution is also used at the same time to clean the membrane of the filtration module. Membrane cleaning during ongoing filtration is either not provided in conventional systems or, as in the ATF systems mentioned at the beginning, is carried out by reversing the flow, the detached covering layer components being rinsed out of the filtration module into the storage tank (bioreactor) with the unfiltrate flow.

Alternatively, it is possible to use pure water (WFI), which is less expensive than a buffer solution, or another suitable liquid as a rinsing liquid instead of a buffer solution.

According to a particular aspect of the invention, backflushing is carried out with substrate, i.e. a nutrient solution intended for cultivating the cells in the bioreactor. Backflushing with substrate is particularly suitable for a fed-batch or continuous process (perfusion process). In such a process, substrate must be delivered into the bioreactor anyway to improve the conditions for cell growth and to compensate for the volume loss caused by the continued removal of filtered protein solution. The substrate can therefore be used simultaneously as a rinsing liquid for cleaning the membrane and as a nutrient solution for cell cultivation.

In the device arrangement according to the invention, a first valve is preferably provided in the return line for selectively blocking the retentate return flow into the storage tank, and is used as a throttle or pressure control valve during filtration to control and regulate the transmembrane pressure (TMP) as the driving force. In addition, a second valve, which is provided in the filtrate line to selectively block the discharge of the filtrate, can be used to prevent the outflow from the filtration module during a backflush process.

A third valve in the backflush line can control the supply of rinsing liquid to the filtration module.

In contrast to the arrangement of the membrane pump in the known ATF systems, the first pump, which is used to maintain the recirculation circuit, is here preferably integrated into the intake line, i.e. it is arranged upstream of the filtration module.

In the event that the device arrangement according to the invention is not to be designed for a batch process but for a fed-batch or continuous process (perfusion process), it is necessary to provide a substrate supply line for supplying substrate into the storage tank, provided that the latter also serves as a bioreactor in which the cells are cultivated.

According to a first variant, this substrate supply line can open directly into the storage tank as a separate line. In this case, a third pump is then provided for conveying the culture medium into the storage tank.

According to an alternative second variant, the substrate supply line and the backflush line open into a common supply line upstream of the second pump, which in turn opens into the otherwise unused permeate-side connection and/or into the permeate-side outlet of the filtration module. This design variant makes it possible to use the second pump, which can in particular be integrated into the common supply line, for both the supply of substrate and the supply of buffer solution. For this reason, a dedicated pump for conveying the substrate can be omitted in this variant. This simplifies the design of the device arrangement, and it can be configured more cost-effectively.

By means of the third valve in the backflush line and an additional fourth valve in the substrate supply line, it is possible to switch between the supply of rinsing liquid and the supply of substrate as required, or to block both lines.

According to a particular embodiment of the device arrangement according to the invention, a sterile filter is arranged in the backflush line or in the common supply line. This can prevent impurities from being introduced into the recirculation circuit from the outside in the course of the rinsing liquid or substrate supply. This is particularly important for perfusion processes which run over a comparatively long period of time, e.g. several weeks. Contamination would have particularly serious consequences here. However, the provision of a sterile filter in the substrate supply line also makes sense in batch or fed-batch processes, as contamination should always be avoided.

To fully exploit the potential of the device arrangement according to the invention, it is advantageous to provide sensors and a control unit for the direct or indirect determination and monitoring of one or more of the following parameters: viscosity; electrical conductivity; flow (in particular recirculation flow and/or filtrate flow); pressure, in particular transmembrane pressure (TMP). In this case, the control unit is set up to initiate and control a backflush depending on whether one or more predetermined limit values of said parameters are reached. In this way, the entire process can be continuously adjusted and controlled in an optimal way. Semi-automatic or even fully automatic control of the process is particularly advantageous if the control unit can, for example, control the individual pumps and valves of the device arrangement. In particular, the parameters can be used to determine optimum times for the rinsing processes with simultaneous dilution, and the duration, pressure and quantity of rinsing liquid for the rinsing processes can also be optimally set to achieve or maintain the desired performance values of the process (filtration performance, degree of dilution, etc.).

The invention also provides a method of separating cells from a culture medium, in particular using a device arrangement according to the invention. The method according to the invention comprises the following steps:
cultivating cells in a culture medium in a bioreactor;
filtering the culture medium in a filtration module having at least one membrane, in particular in a hollow fiber module, the culture medium passing through a recirculation circuit;
rinsing the membrane using a rinsing liquid during filtration, the rinsing liquid being in particular a buffer solution or water or substrate, and rinsing liquid being introduced into the recirculation circuit by the rinsing.

According to the above explanations, “rinsing during filtration” means that the rinsing processes are embedded in the superordinate filtration step. Depending on requirements, the recirculation circuit can be completely interrupted or maintained to a limited extent for a rinsing process. While the permeate discharge should definitively be closed during a rinsing process, the recirculation pump (first pump) can continue to run, possibly with reduced output, or even be completely switched off. The retentate return to the storage tank should be open during the rinsing process, at least if the circulation pump was not stopped during backflush. During the actual filtration, the valve arranged in the retentate return serves to regulate the transmembrane pressure and can be partially or temporarily closed.

To permit an independent supply of rinsing liquid into the filtration module, it is preferably provided that it is conveyed via a separate line into one or more permeate-side connection(s) of the filtration module, which is/are separate from the unfiltrate inlet of the filtration module, into which the culture medium to be filtered is conveyed from the storage tank.

For optimum adjustment and control of the process, including the simultaneous rinsing and dilution processes, it is advisable to determine and monitor one or more of the following parameters at least during filtration: viscosity; electrical conductivity; flow (especially recirculation flow and/or filtrate flow); pressure, especially transmembrane pressure. As already described above, a backflush is then initiated and controlled depending on whether one or more predetermined limit values of said parameters are reached.

The rinsing step can be carried out several times in succession and/or repeated cyclically during the superordinate filtering step, wherein the time intervals between the rinsing processes can vary, in particular depending on the parameters continuously determined by the sensors.

The rinsing liquid can also be varied during the rinsing processes. It is possible to switch between a buffer solution, pure water and a substrate or at least between two of these liquid media either according to a predetermined scheme or according to requirements determined on the basis of the aforementioned parameters. In principle, other rinsing liquids can also be included.

When using the method according to the invention in a batch process, the cultivating and filtering steps are carried out one after the other.

In contrast thereto, in the case of a fed-batch or perfusion process, the cultivating and filtering steps are carried out at least partially simultaneously.

### BRIEF DESCRIPTION OF THE DRAWINGS

Further features and advantages of the invention will become apparent from the following description and from the accompanying drawings, to which reference is made and in which:

FIG. 1 shows a schematic set-up of a device arrangement according to a first embodiment of the invention;

FIG. 2 shows a schematic set-up of a device arrangement according to a second embodiment of the invention; and

FIG. 3 shows a schematic set-up of a device arrangement according to a third embodiment of the invention.

### DETAILED DESCRIPTION OF THE INVENTION

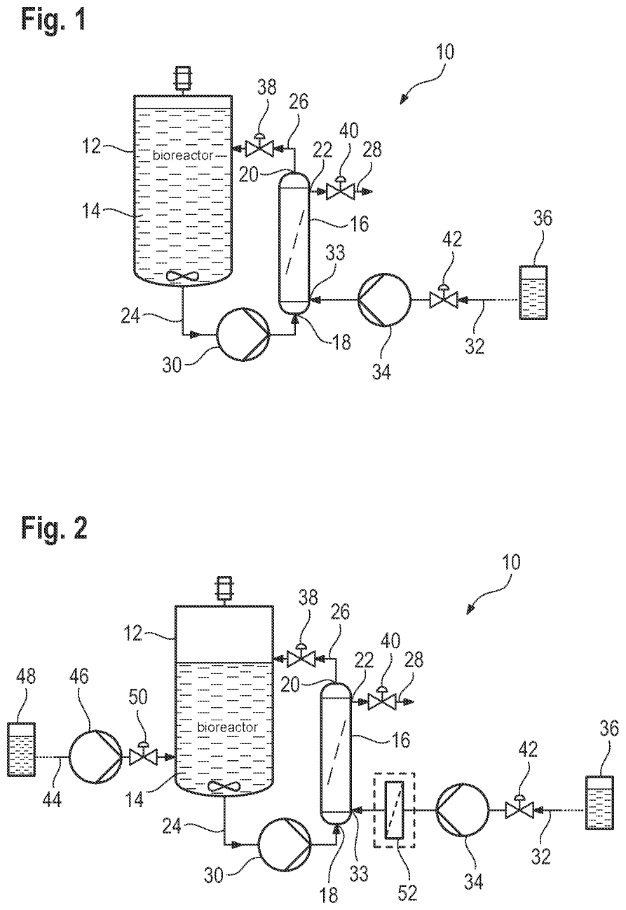

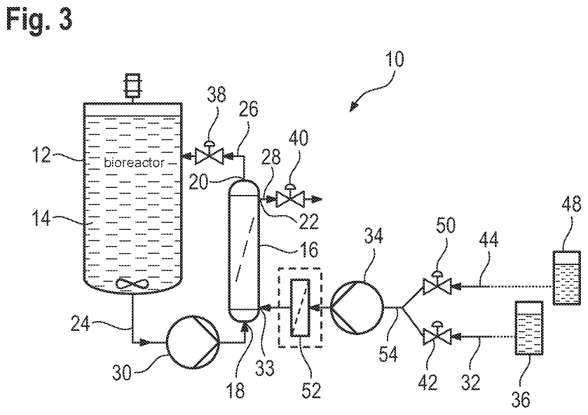

In the following, three different set-ups of a device arrangement 10 by means of which cells are separated from a culture medium in the context of a biopharmaceutical fermentation process are described as examples on the basis of FIGS. 1 to 3. The same reference symbols are used for corresponding components, only the essential differences to the set-up shown in FIG. 1 being discussed with regard to the set-ups shown in FIGS. 2 and 3.

According to the set-up shown in FIG. 1, the device arrangement 10 comprises a storage tank 12 for a culture medium 14 and a filtration module 16, by means of which cells can be separated from the culture medium 14 to obtain a target protein. The storage tank 12 is a bioreactor in which the fermentation process for cultivating the cells takes place.

The filtration module 16 here is a hollow fiber module having at least one membrane which is adapted to be overflowed, i.e. the filtration module 16 is designed for cross-flow filtration. Accordingly, the filtration module 16 has at least three connections, namely an unfiltrate inlet 18 for the unfiltrate from the storage tank 12, a retentate-side outlet 20, and a permeate-side outlet 22. In many hollow fiber modules, a further permeate-side connection 33 is provided, which is not used in some conventional filtration applications and is therefore closed with a plug. The special use of this connection 33 in conjunction with the invention will be discussed in more detail later.

An intake line 24 connects the storage tank 12 to the inlet 18 of the filtration module 16. A return line 26 connects the retentate-side outlet 20 of the filtration module 16 to the storage tank 12. A filtrate line 28 is connected to the permeate-side outlet 22 of the filtration module 16.

A first pump 30 by means of which a recirculation circuit of the unfiltrate can be maintained, is integrated into the intake line 24. In this recirculation circuit, culture medium 14 is conveyed from the storage tank 12 through the intake line 24 to the unfiltrate inlet 18 of the filtration module 16. In the filtration module 16, the culture medium 14 flows over the membrane, which is permeable to the target protein, but not to the cells. While the solution freed of cells (permeate or filtrate) is discharged via the permeate-side outlet 22, the retentate is returned to the storage tank 12 via the outlet 24 and the return line 26. The first pump 30 could in principle also be integrated into the return line 26 for this purpose, in which case no positive transmembrane pressure could be built up.

Furthermore, a backflush line 32 leads to a permeate-side connection 33 of the filtration module 16. A second pump 34 is integrated into the backflush line 32, by means of which a rinsing liquid can be conveyed from a reservoir 36 to the permeate-side connection 33 of the filtration module 16. The reservoir 36 is filled in particular with a buffer solution or water for injection purposes (WFI); however, another suitable rinsing liquid can also be used.

A first valve 38 is integrated into the return line 26, by means of which the retentate return flow into the storage tank 12 can be blocked or reduced. A second valve 40 is integrated into the filtrate line 28. This second valve 40 can be used to block or reduce the discharge of the filtrate (cell-free culture medium). A third valve 42 integrated into the backflush line 32 serves to selectively block or reduce the supply of rinsing liquid to the permeate-side connection 33 of the filtration module 16.

Preferably, all components of the device arrangement 10, in particular the filtration module 16, the lines 24, 26, 28, 32 and the valves 38, 40, 42 as well as at least the parts of the pumps 30, 34 which come into contact with the medium or rinsing liquid are designed as single-use components. This means that these components consist primarily of plastics adapted to be sterilized or autoclaved. The components can be pre-assembled before use, sterilized and connected to the bioreactor in a sterile manner using suitable sterile connectors. After a single use, all single-use components are completely disposed of.

The set-up shown in FIG. 1 is particularly suitable for a so-called batch process, in which no further addition of substrate is provided after the initial filling of the bioreactor, which also serves as a storage tank 12, and the start of cultivation.

The bioreactor is filled to the target volume with substrate and inoculated with cells (inoculation). As soon as the optimum cell or protein concentrations (titers) are reached, the process step of cell separation or collection of the target protein takes place. The protein yield should be as high as possible, and the cells (biomass) should be completely separated from the protein-containing solution.

With conventional technologies such as centrifugation and/or depth filtration, protein yield rates of only 85% or even less are generally achieved. At the same time, dilution is unavoidable due to process-related conditions. Depending on the process conditions and the intended yield, this can be up to 30%. Both parameters, yield and dilution, can only be influenced to a limited extent in conventional processes. With the clear upward trend in biotechnological fermentation processes towards intensification and increasing titers and thus yields, the amount of material used in these conventional separation processes is increasing exorbitantly and is now becoming more and more unattractive in economic terms.

The set-up of the device arrangement 10 described above and the procedure described below during filtration open up new possibilities for optimization in this respect, the required space and the technological effort being not so strongly dependent on the titer and cell concentration.

According to the method proposed here, filtration takes place in the recirculation circuit already described above, in which the culture medium 14 repeatedly passes through the filtration module 16. From a desired and possibly previously determined operating point (in particular with regard to viscosity, flow (recirculation flow and/or filtrate flow), transmembrane pressure, dilution), the filtration or concentration is interrupted by cyclic backflushing. For this purpose, the second pump 30 is used to convey rinsing fluid from the reservoir 36 to the permeate-side connection 33 of the filtration module 16. The rinsing liquid flowing through the membrane onto the retentate side ensures that the membrane is cleaned, as will be explained in more detail below.

A buffer solution which is anyway used in the context of the fermentation process is particularly suitable as a rinsing liquid. Such a buffer solution therefore behaves neutrally and only increases the volume in the recirculation circuit, which is precisely desirable in view of the increasing viscosity of the culture medium due to the removal of the permeate. Therefore, the (partial) process steps “dilution of the culture medium” and “cleaning of the membrane in the filtration module 16” can be combined into a single step (“2 in 1”) during filtration.

Alternatively, rinsing with pure water (WFI) or a switch between buffer solution and water is also possible.

Depending on which differential pressure conditions are to be achieved, the first pump 30 can continue to run, be throttled or even switched off during backflushing. During the backflushing process, the second valve 40 in the filtrate line 28 is generally closed, and the first valve 38 in the return line 26 is open. A differential pressure is thus generated on the membrane surface, which counteracts the filtration flow, causing the covering layer on the membrane to be completely or partially detached. This effectively counteracts blocking of the filter (fouling), which leads to at least a temporary increase in the previously reduced filtration performance.

After backflushing, filtration is continued. The backflushing process can be repeated as often as desired or carried out as long as desired until the respective limit values in terms of yield, dilution and/or process time are reached.

By using suitable sensors from known cross-flow filtration systems, which can be integrated into the device arrangement 10 as single-use sensors, it is possible to permanently measure the filtration performance and use a control unit to determine the ideal parameters (in particular time, duration, volume, pressure, speed) of the backflush and adjust them accordingly or change and optimize them in the process, so that the filtration performance is maintained in the best possible way over the entire process duration.

In the set-up of the device arrangement 10 shown in FIG. 2, a substrate supply line 44 is also provided, which opens into the storage tank 12. A third pump 46 is integrated into the substrate supply line 44 so that substrate, e.g. from a tank 48, can be conveyed into the bioreactor. The supply of substrate can be selectively blocked or reduced by means of a fourth valve 50 integrated into the substrate supply line 44. Optionally, a sterile filter 52 can be integrated into the backflush line 32. The provision of such a sterile filter also makes sense in the substrate supply line 44 to reliably maintain sterility in the bioreactor from this side.

The set-up shown in FIG. 2 is particularly suitable for a so-called fed-batch or continuous process (perfusion process), in which fresh substrate is added to the bioreactor either in one step, cyclically or continuously in the course of the fermentation process to control and positively influence the living conditions for the cells and thus the protein yield. While in the fed-batch process the cells are separated from the target protein in a single step at the end of the cultivation process, this step takes place in the perfusion process during the cultivation process itself, quasi in a semi-continuous or continuous mode. At the end of the fed-batch process or in the course of perfusion, the conventional technologies of centrifugation and/or depth filtration or the cross-flow filtration of interest here are used for cell separation. Partial quantities are obtained in the course of cultivation, followed at the end by a final up-concentration to increase the yield.

The set-up of the device arrangement 10 shown in FIG. 2 and the way of proceeding in the cell separation as described below also open up new possibilities for optimization with regard to fed-batch and perfusion processes, the required space and the technological effort being not so strongly dependent on the titer and cell concentration.

The addition of culture medium 14 during the fed-batch or perfusion process and the dilution thereof with buffer solution and/or pure water in the context of the optimization of the ongoing process can also be combined with backflushing for membrane purification. As described above, filtration is interrupted from the desired operating point (in particular with regard to viscosity, flux performance, dilution) by cyclic backflushing and thus cleaning of the membrane surface with a suitable buffer solution and/or pure water. However, the substrate is supplied directly into the bioreactor via the separate substrate supply line 44 and the third pump 46, while the supply of buffer solution and/or pure water—and the simultaneous membrane backflushing—is carried out into the permeate-side connection 33 of the filtration module 16 via the backflush line 32 and the second pump 34.

FIG. 3 shows an alternative set-up of the device arrangement 10, which is also suitable for a fed-batch or perfusion process. The set-up largely corresponds to the set-up shown in FIG. 2, but differs in the type of substrate supply, as described briefly below.

Here, the substrate supply line 44 does not open directly into the bioreactor, the substrate supply line 44 and the backflush line 32 both open into a common supply line 54, which in turn opens into the permeate-side connection 33 of the filtration module 16. The second pump 34 and the optional sterile filter 52 are integrated into this common supply line 54.

With this set-up, it is possible to realize the supply of the substrate and the rinsing liquid with the same pump, namely the second pump 34. In this set-up variant, the substrate is not supplied directly into the bioreactor, but indirectly via the permeate-side connection 33 of the filtration module 16, through the membrane and via the return line 26. By switching the third valve 42 and the fourth valve 50 accordingly, it is possible to selectively supply the substrate from the medium tank 48 or the rinsing liquid from the reservoir 36, depending on the (partial) process step, with the backflushing and the associated membrane cleaning taking place simultaneously with the supply.

In principle, it is possible in all of the set-ups described above to convey the rinsing liquid into the filtration module 16 not (only) via the permeate-side connection 33, but instead or additionally also via the permeate-side outlet 22, which is actually intended for filtrate discharge. For this purpose, a suitable connection with valves must be provided, which can separate the rinsing liquid supply from the filtrate discharge at the permeate-side outlet 22. In principle, any permeate-side connection of the filtration module 16 (alone or in combination with others) can be used for backflushing. Although such solutions are not explicitly shown individually in the figures, they follow the same basic idea as the embodiments described above.

In all of the set-ups described above, cell separation can be implemented as a completely closed one-way process step due to the availability of the necessary components. The pumps 30, 34, 46 (at least the parts thereof which are in contact with the medium or rinsing liquid), the lines 24, 26, 28, 32, 44, 54 as well as the valves 38, 40, 42, 50 and the sensors are made of sterilizable single-use materials.

As also described above, the filtration performance can be permanently measured in all process variants, and the ideal parameters (in particular time, duration, volume, pressure, speed) of the backflush can be determined using a control unit and set accordingly or changed and optimized in the process, to optimize yield and efficiency. Known single-use sensors for measuring or determining the pressure (in particular transmembrane pressure), flow, viscosity, conductivity and their changes in the course of the process are already used here.

### LIST OF REFERENCE NUMERALS

10 device arrangement
12 storage tank
14 culture medium
16 filtration module
18 unfiltrate inlet
20 retentate-side outlet
22 permeate-side outlet
24 intake line
26 return line
28 filtrate line
30 first pump
32 backflush line
33 permeate-side connection
34 second pump
36 reservoir
38 first valve
40 second valve
42 third valve
44 substrate supply line
46 third pump
48 medium tank
50 fourth valve
52 sterile filter
54 common supply line

1. A device arrangement for separating cells from a culture medium in a biopharmaceutical process, comprising:
a storage tank for the culture medium in a bioreactor,
a filtration module, being a hollow fiber module, having at least one membrane adapted to be overflowed,
an intake line which connects the storage tank to an unfiltrate inlet of the filtration module for supplying the culture medium into the filtration module,
a return line which connects a retentate-side outlet of the filtration module to the storage tank for returning retentate into the storage tank,
a filtrate line which is connected to a permeate-side outlet of the filtration module for discharging filtrate,
a first pump for maintaining a recirculation circuit,
a backflush line which leads to a permeate-side connection and/or to the permeate-side outlet of the filtration module for supplying a rinsing liquid into the filtration module, and
a second pump for conveying the rinsing liquid into the filtration module.

2. The device arrangement according to claim 1, characterized in that the rinsing liquid is one of the following: buffer solution; water; or substrate.

3. The device arrangement according to claim 1, characterized by a first valve in the return line for selectively blocking a return flow of the retentate into the storage tank.

4. The device arrangement according to claim 3, characterized by a second valve in the filtrate line for selectively blocking the discharge of the filtrate.

5. The device arrangement according to claim 4, characterized by a third valve in the backflush line for selectively blocking the supply of the rinsing liquid to the filtration module.

6. The device arrangement according to claim 1, characterized in that the first pump is integrated into the intake line.

7. The device arrangement according to claim 5, characterized by a substrate supply line for supplying substrate into the storage tank.

8. The device arrangement according to claim 7, characterized in that the substrate supply line opens directly into the storage tank, and in that a third pump is provided for conveying the substrate into the storage tank.

9. The device arrangement according to claim 7, characterized in that the substrate supply line and the backflush line open upstream of the second pump into a common supply line, which in turn opens into the permeate-side connection and/or into the permeate-side outlet of the filtration module.

10. The device arrangement according to claim 7, characterized by a fourth valve in the substrate supply line for selectively blocking the supply of substrate.

11. The device arrangement according to claim 9, characterized by a sterile filter integrated in the backflush line or in the common supply line.

12. The device arrangement according to claim 1,
characterized by sensors and a control unit for directly or indirectly determining and monitoring one or more of the following parameters: viscosity; electrical conductivity; recirculation flow and/or filtrate flow; or transmembrane pressure;
the control unit being set up to initiate and control a backflush depending on whether one or more predetermined limit values of said parameters are reached.

13. A method of separating cells from a culture medium, using a device arrangement according to claim 1, the method comprising steps of:
cultivating cells in a culture medium in a bioreactor;
filtering the culture medium in a filtration module having at least one membrane, being a hollow fiber module, the culture medium passing through a recirculation circuit;
rinsing the membrane using a rinsing liquid during filtration, the rinsing liquid being a buffer solution or water or substrate, and rinsing liquid being introduced into the recirculation circuit by the rinsing.

14. The method according to claim 13, characterized in that the rinsing liquid is conveyed via a separate line into one or more permeate-side connection(s) of the filtration module.

15. The method according to claim 13, characterized in that
one or more of the following parameters are determined and monitored at least during filtration: viscosity; electrical conductivity; recirculation flow and/or filtrate flow; or transmembrane pressure; and
in that a backflush is initiated and controlled depending on whether one or more predetermined limit values of said parameters are reached.

16. The method according to claim 13, characterized in that the rinsing step is carried out several times in succession.

17. The method according to claim 13, characterized in that the rinsing step is repeated cyclically during filtration.

18. The method according to claim 17, characterized in that the repeated rinsing step is carried out with different rinsing liquids.

19. The method according to claim 13, characterized in that the cultivating and filtering steps are carried out one after the other.

20. The method according to claim 13, characterized in that the cultivating and filtering steps are carried out at least partially simultaneously.
